(19) United States
(12) Patent Application Publication
(10) Pub. No.: US 2019/0217262 A1
(43) Pub. Date: Jul. 18, 2019

(54) Bone Cement Mixing Apparatus and Method Therefor

(71) Applicant: Sky Light Medical Limited, Glasgow (GB)

(72) Inventor: Colin McKay, Glasgow (GB)

(73) Assignee: Sky Light Medical Limited, Glasgow (GB)

(21) Appl. No.: 16/326,351

(22) PCT Filed: Aug. 16, 2017
(86) PCT No.: PCT/GB2017/052417

§ 371 Date: Feb. 18, 2019

(30) Foreign Application Priority Data
Aug. 16, 2016 (GB) 1614011.3

(51) Int. Cl. B01F 11/00; B01F 15/00; A61B 17/88

(52) U.S. Cl. CPC B01F 11/0054; B01F 15/00519; B01F 2215/0029; A61B 17/8833; B01F 15/00538

(57) ABSTRACT

A bone cement mixing apparatus for mixing components of bone cement is described. The apparatus comprises a mixer unit, the mixer unit having a drive mechanism and a mixing cylinder comprising a housing and a mixing mechanism, the mixing mechanism adapted, in use, to mix components of bone cement contained within the housing. The drive mechanism is adapted to create rotational movement of the mixing mechanism with respect to the mixing cylinder housing and to create axial movement of the mixing mechanism with respect to the mixing cylinder housing.

### FIELD

The present invention relates to mixing apparatus and a method of mixing different components. In particular, the present invention relates to bone cement mixing apparatus along with a method for mixing material to fill bone cavities.

### BACKGROUND

In orthopaedic surgery acrylic bone cement is used as a grout to fixate implants that restore joint function. The cement is composed of a powder polymer (polymethylmethacrylate) contained in a paper packet and liquid monomer (methylmethacrylate) contained in a glass ampoule. When combined together in a mixing cylinder and mixed together the two components polymerise and can be injected from the mixing cylinder via means of a cement gun into or onto the appropriate anatomy.

It has been found that the quality of mixing is essential to the performance of the finished cement. The life of the cement improves as the porosity of the cement decreases. Additionally, poor mixing does not fully mix the polymer powder into the liquid monomer and in the event of fracture of the cement, this unmixed polymer powder can lead to osteolysis.

Conventional mixing systems are generally manual and involve pushing and rotating a plunger through the components. This leads to a largely uneven mix with high porosity. Furthermore, it can be tiring for the person creating the mix and lead to conditions like repetitive strain injury.

It is known to attach the plunger to a medical drill to create a better mix by faster rotation of the plunger but the quality of the mixed cement is still affected by the rate at which the user pushes and pulls the plunger through the components.

### SUMMARY

According to a first aspect of the present invention there is provided a mixing apparatus for mixing components of bone cement, the apparatus comprising:

a mixer unit, the mixer unit having a drive mechanism; and

a mixing cylinder comprising a housing and a mixing mechanism, the mixing mechanism adapted, in use, to mix components of bone cement contained within the housing;

wherein the drive mechanism is adapted to create rotational movement of the mixing mechanism with respect to the mixing cylinder housing and to create axial movement of the mixing mechanism with respect to the mixing cylinder housing.

In at least one embodiment of the invention, providing a drive mechanism which both rotates and axially translates the mixing mechanism with respect to the mixing cylinder housing, provides a finished product which is more homogenously mixed with lower porosity. Initial findings have shown that a low porosity mix can be achieved using the mixing apparatus without the need for applying a vacuum line to the mixer apparatus housing in certain circumstances.

Furthermore, a mixing apparatus of this type is easier to use than conventional manual systems resulting in less risk of repetitive strain injury and other injuries associated with manual work.

The drive mechanism may comprise a first drive motor and a second drive motor, the first drive motor being adapted to create rotational movement of the mixing mechanism with respect to the mixing cylinder housing and the second mixer drive motor being adapted to create axial movement of the mixing mechanism with respect to the mixing cylinder housing. In at least one embodiment of the present invention, providing a mixing apparatus with separate motors for rotating and driving the mixer mechanism reduces the load on each motor, increasing the life of the unit and reducing the cost of the unit.

The mixer unit may be electrically powered.

The mixer unit may be adapted to be sterilised in an autoclave. Being able to sterilise the unit in an autoclave reduces the risk of bringing infection into the operating theatre.

The mixer unit may be powered by at least one battery. Powering the mixer unit by one or more batteries makes the mixer unit fully portable, free from the constraints of requiring a fixed electrical power supply, further facilitating the ability to sterilise the unit.

In at least one embodiment, the mixer unit drive mechanism is battery powered.

In at least one embodiment, in which the drive mechanism comprises a first and second drive motor, at least one of the drive motors is battery powered.

In a preferred embodiment, both the drive motors are battery powered.

The/each battery may be replaceable.

The/each battery may be removable. Providing a removable battery allows the mixer unit to be sterilised in an autoclave.

The mixer unit may comprise a battery compartment.

The battery compartment may be defined by a mixer unit body.

The battery compartment may be an opening defined by the mixer unit body. Providing a battery compartment as an opening in the mixer unit body, as opposed to a covered battery compartment, allows the mixer unit to be fully sterilised in an autoclave.

The mixing mechanism may comprise a shaft and a head.

The head may comprise a disc, the disc being mounted a right angles to a first end of the shaft.

The head and the shaft may be rotationally fixed.

The mixing mechanism may be adapted to rotate about the shaft axis.

The mixing mechanism may be adapted to move axially in a direction parallel to the shaft axis.

The disc may define a plurality of apertures or openings. Apertures assist the movement of the disc through the components of bone cement as they are mixed.

At least some of the openings may be defined by an edge of the disc.

The mixing mechanism shaft may be attached at a second end to the drive mechanism.

The drive mechanism may be adapted to rotate the mixing mechanism shaft.

Where the drive mechanism comprises a first drive motor and a second drive motor, the first drive motor may rotate the mixing mechanism shaft.

In this embodiment, the second drive motor may be adapted to rotate a threaded bar, the threaded bar defining a threaded profile.

In this embodiment, the threaded bar may be cooperatively attached to a coupling defining a complimentary threaded profile to the threaded bar.

The threaded bar may be rotationally fixed with respect to the coupling and adapted to cooperate with coupling such that rotational movement of the threaded bar creates linear movement of the coupling along the threaded bar.

The mixing apparatus may comprise limiting means adapted to limit the axial movement of the mixing mechanism within the mixing cylinder.

The mixing mechanism may be adapted to actually translate between two predetermined locations in the mixing cylinder.

A first predetermined location may be proximal the mixer unit and a second predetermined location may be distal the mixer unit.

In travelling between the first predetermined location on the second predetermined location, the mixing mechanism may be adapted to rotate multiple times.

According to a second aspect of the present invention there is provided a mixing apparatus for mixing components of bone cement, the apparatus comprising:

a mixer unit, the mixer unit having at least one motor; and

a mixing cylinder comprising a housing and a mixing mechanism, the mixing mechanism adapted to mix components of bone cement contained within the housing, the at least one motor being adapted to create relative movement between the housing and the mixing mechanism;

wherein the at least one motor is battery-powered.

According to a third aspect of the present invention is provided a method of mixing components of bone cement, the method comprising the steps of:

attaching a mixing cylinder to a mixer unit;

introducing components of bone cement to the mixing cylinder;

actuating the mixer unit to mix the components of bone cement by creating relative movement in rotational and axial direction of a mixing cylinder mixing mechanism with respect to a mixing cylinder housing;

removing the mixing cylinder from the mixer unit.

The step of actuating the mixer unit to mix the components of bone cement by creating relative movement in rotational and axial direction of a mixing cylinder mixing mechanism with respect to a mixing cylinder housing may further comprise the steps of:

actuating a first mixer unit drive motor to create rotational movement of the mixing cylinder mixing mechanism with respect to the mixing cylinder housing; and

actuating a second mixer unit drive motor to create axial movement of the mixing cylinder mixing mechanism with respect to the mixing cylinder housing.

The step of attaching the mixing cylinder to the mixer unit may further comprise the steps of:

attaching a mixing cylinder mixing mechanism shaft to a mixer unit drive mechanism; and

attaching a mixing cylinder housing to a mixer unit body.

The method may further comprise the step of inserting a battery into a mixer unit battery compartment, the battery being provided to power the mixer unit.

Additionally, the method may comprise the further step of providing a battery in a sterile pack and removing the battery from the pack.

It will be understood that features listed as non-essential with respect any of the above listed aspects of the invention may be equally applicable to any of the other aspects of the invention but have not been repeated for brevity.

### BRIEF DESCRIPTION OF THE DRAWINGS

An embodiment present invention will be described with reference to the accompanying drawings in which:

FIG. 1 is a schematic view of a mixing apparatus according to a first embodiment of the present invention;

FIG. 2, comprising FIG. 2a and FIG. 2b, are perspective views of a mixer unit from the mixing apparatus of FIG. 1;

FIG. 3 is a plan view of a mixing mechanism disc from the mixing apparatus of FIG. 1;

FIGS. 4 to 13 are schematic views showing the operation of the mixing apparatus of FIG. 1.

### DETAILED DESCRIPTION OF THE DRAWINGS

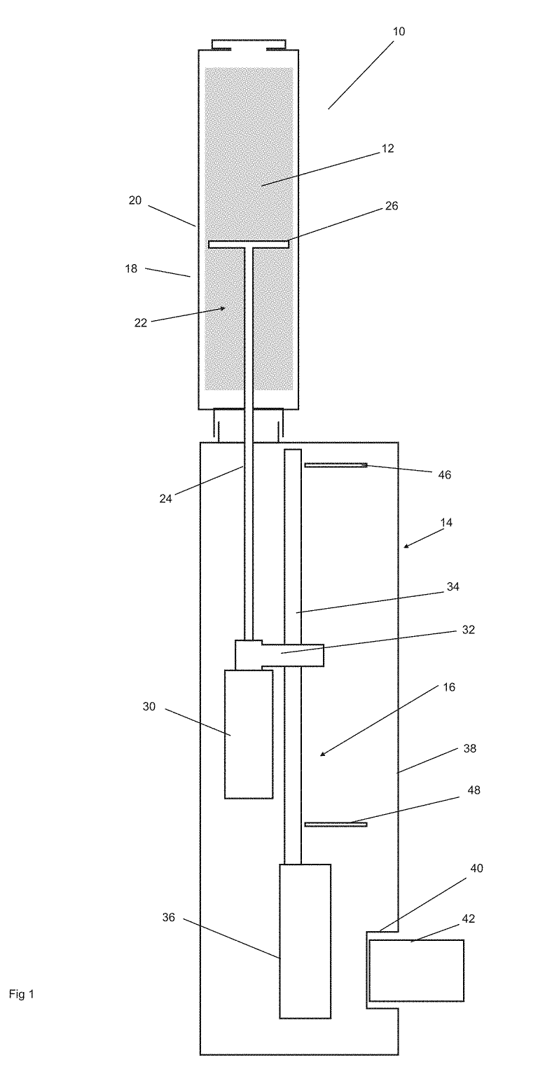

Reference is first made to FIG. 1, a schematic view of a mixing apparatus, generally indicated by reference numeral 10, for mixing components of bone cement 12 according to a first embodiment of the present invention.

The mixing apparatus 10 comprises a mixer unit 14 having a drive mechanism 16. The mixing apparatus 10 further comprises a mixing cylinder 18 comprising a housing 20 and a mixing mechanism 22, the mixing mechanism 22 adapted to mix the components of bone cement 12 contained within the housing 20.

The drive mechanism 16 is adapted to create axial and rotational movement of the mixing mechanism 22 with respect to the mixing cylinder housing 20.

Such an arrangement permits a thorough mixing of the components of bone cement 12 in the mixing cylinder housing 20, resulting in a homogeneous bone cement of low porosity.

The mixing mechanism 22 comprises a shaft 24 and a head 26 in the form of a disc 28. The shaft 24 and the head 26 are rotationally fixed, and the shaft 24 is attached to a first drive mechanism motor 30, the first drive mechanism motor 30 adapted to rotate the shaft 24 and head 26 to mix the components of bone cement 12 contained within the housing 20.

The mixing mechanism 22 and the first drive mechanism motor 30 are connected, in turn, through a coupling 32 to a lead screw 34. The lead screw 34 is in a threaded relationship with an internal thread on a coupling aperture (not visible) defined by the coupling 32.

The lead screw 34 is attached to a second drive mechanism motor 36 which is adapted to rotate the lead screw 34. The second drive mechanism motor 36 and the lead screw 34 are fixed with respect to a mixer unit housing 38, and rotation of the lead screw 34 causes axial movement of coupling 32 along the lead screw 34 which in turn creates axial movement of the mixing mechanism 22 within the mixing cylinder housing 20. This axial movement of the mixing mechanism 22 further aids mixing of the components of bone cement 12 contained within the housing 20.

The mixing apparatus 10 further comprises a power supply 40 in the form of a battery 42. The battery 42 is removable from the mixer unit 14 and particularly from a battery recess 44 defined by the mixer unit housing 38.

The mixing apparatus further includes an upper limit switch 46 and a lower limit switch 48, which will be discussed in due course.

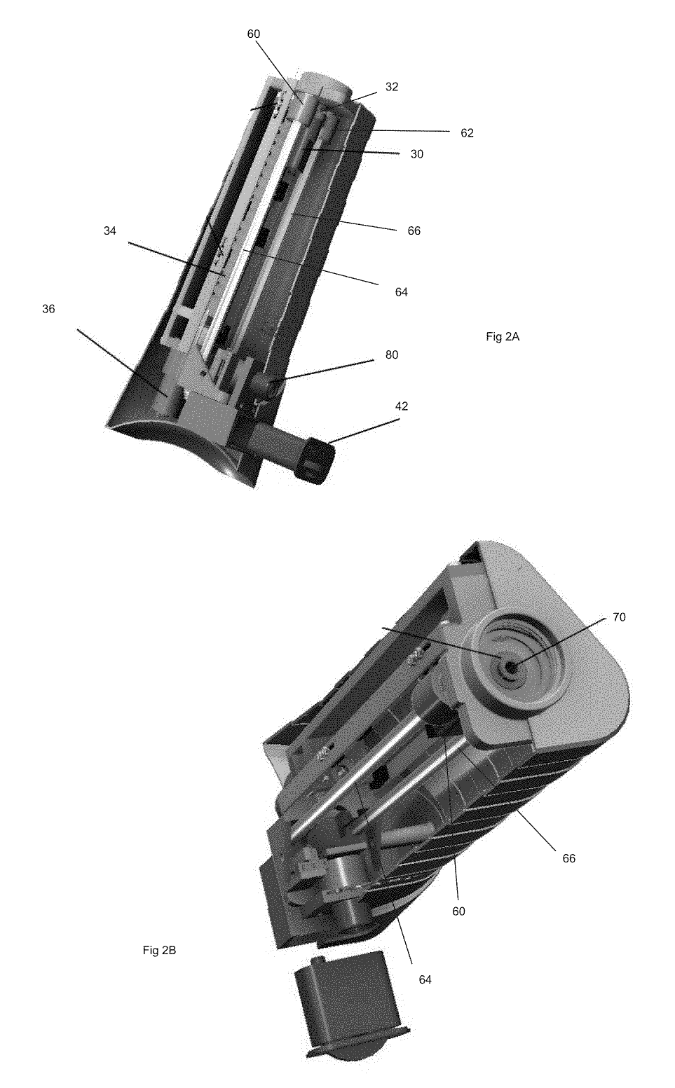

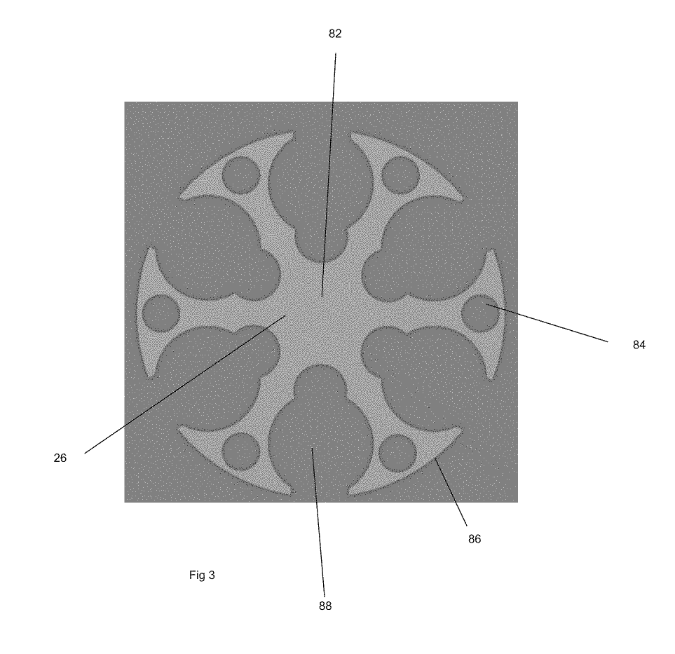

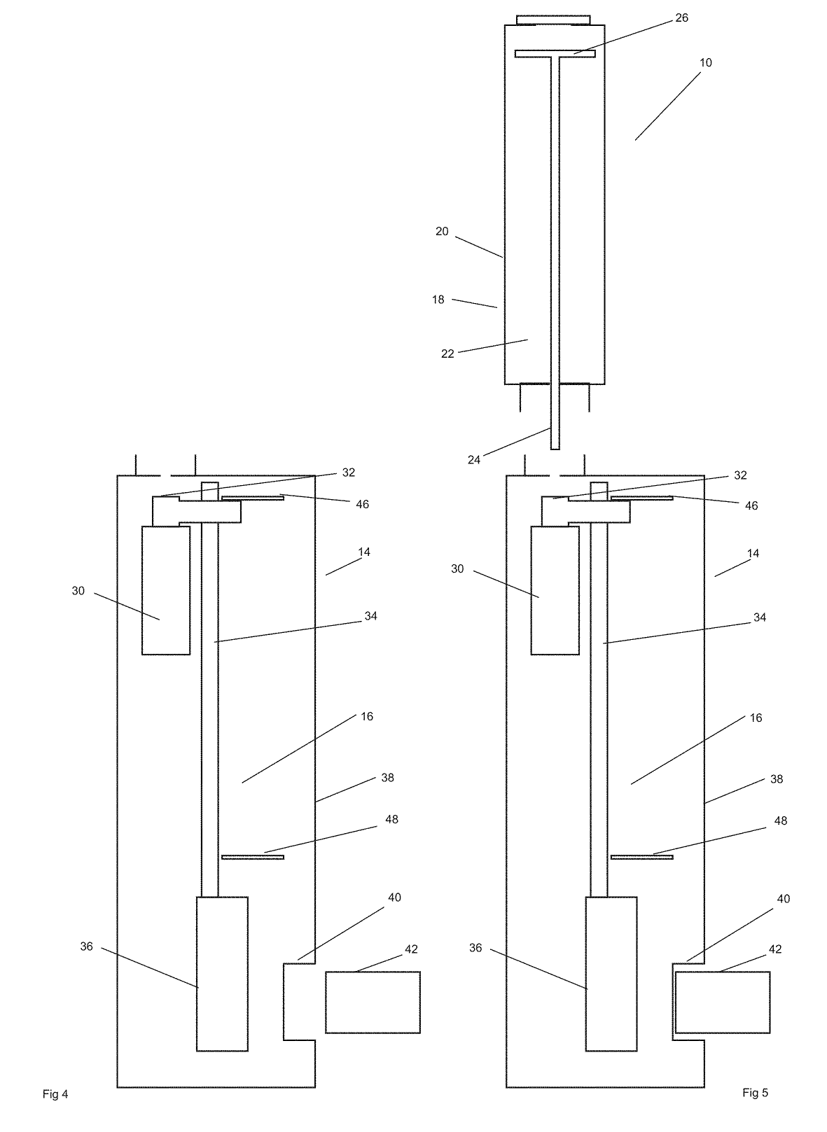

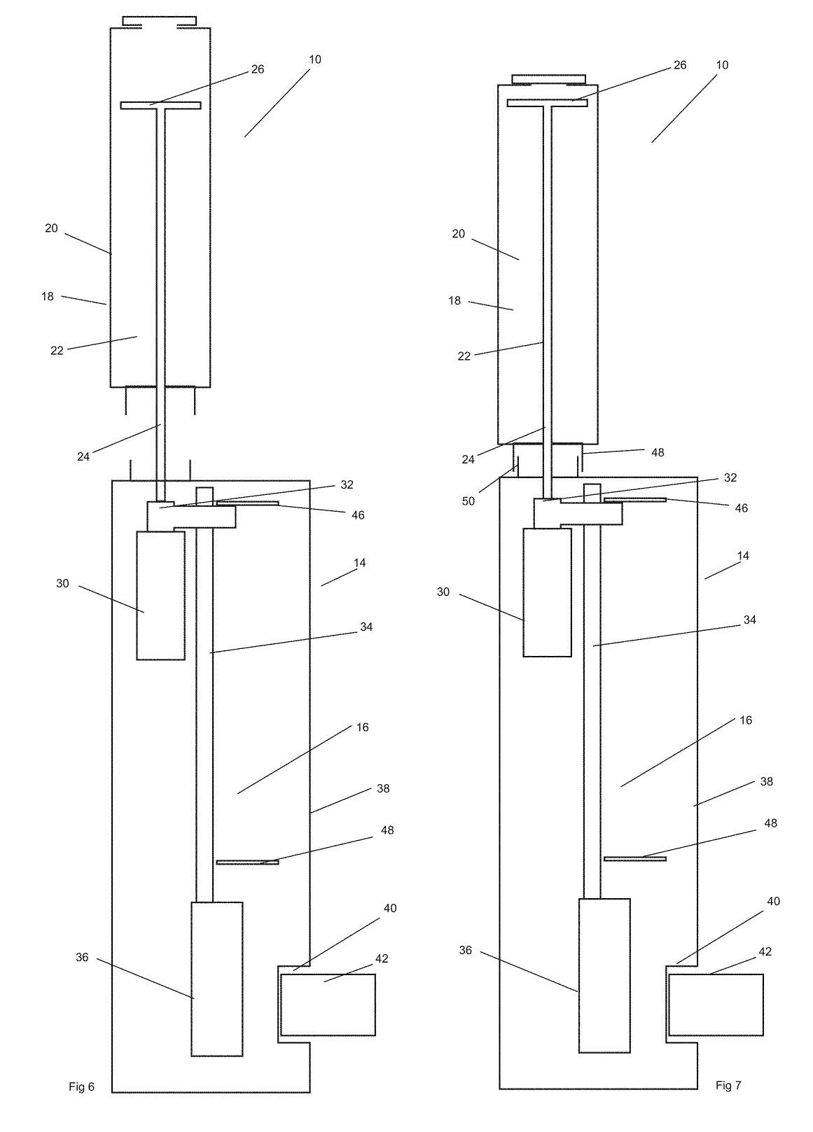

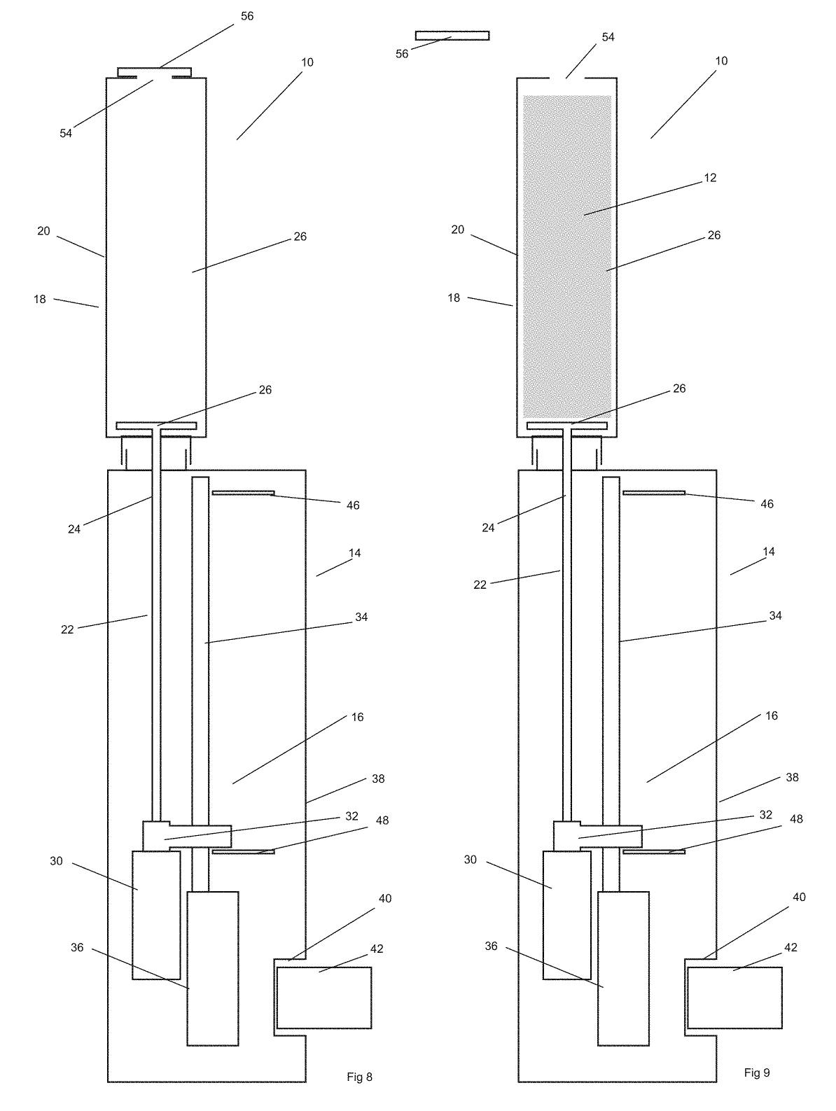

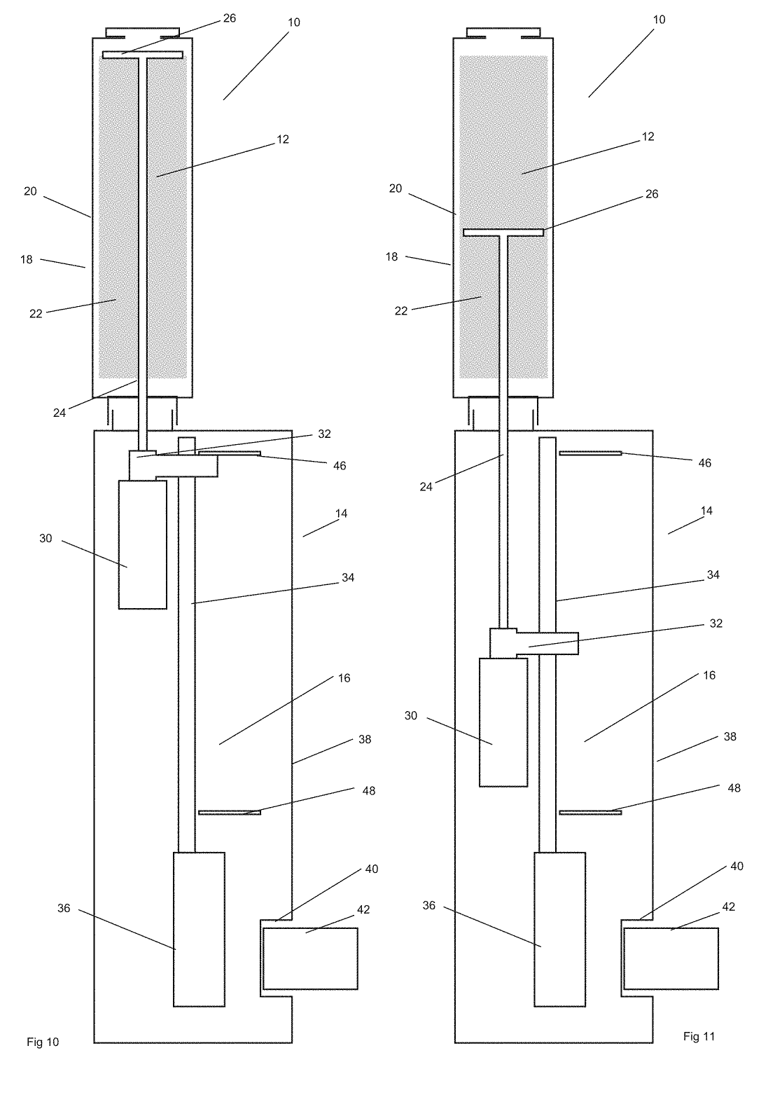

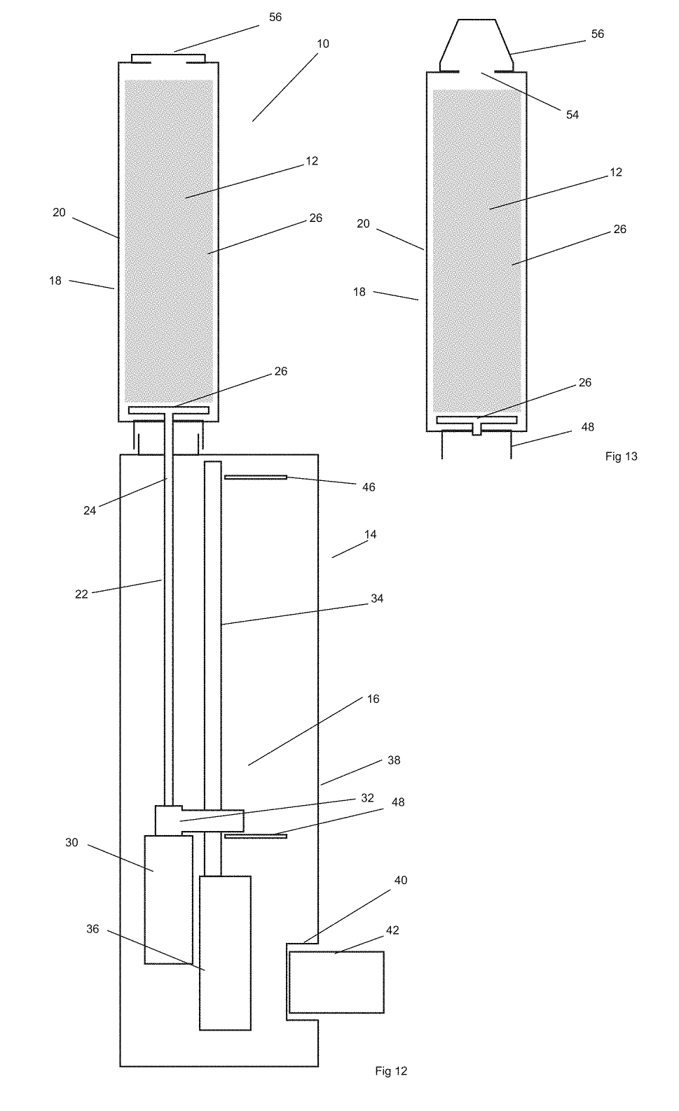

Referring to FIG. 2A, a first perspective view of the mixer unit 14 of the mixing apparatus 10 of FIG. 1, it can be seen the coupling 32 is also attached to a pair of slides 60, 62. The slides 60, 62 are adapted to slide along rods 64, 66. The purpose of the slides 60, 62 and rods 64, 66 is to prevent rotation of the coupling 32 with the rotating lead screw 34 and to maintain the direction of travel of the mixing mechanism 22 along the axis of the mixing cylinder 18. For clarity of presentation, the slides 60, 62 and rods 64, 66 shown in FIG. 2 are not shown in any of the other Figures. Also visible in FIG. 2A is a start button 80 for starting and stopping the drive mechanism 18, and particularly the motors 30, 36.

Operation of the mixing apparatus 10 will now be described with reference to a series of schematic drawings number as FIGS. 4 to 13.

Reference first made to FIG. 4. As the battery 42 is separate from the mixer unit 14, the mixer unit 14 can be sterilised in an autoclave. In surgery, the sterile mixer unit 14 can be brought into theatre along with the sterile battery 42.

In theatre, the sterile battery 42 is removed from its packaging and inserted into a battery recess 44 defined by the mixer unit housing 38.

FIG. 5 shows the mixing cylinder 18 being brought into position adjacent the mixer unit 14.

As shown in FIG. 6, the mixing mechanism shaft 24 is fed through an aperture 70 (visible in FIG. 2B, a second perspective view of the mixer unit 14 of the mixing apparatus 10 of FIG. 1) defined by the mixer unit housing 38 and into engagement with the coupling 32 and first drive motor 30, to which the shaft 24 is fixed.

Referring to FIG. 7, the cylinder housing 20 is then fixed to the mixer unit body 38 by means of a threaded engagement between a mixing cylinder boss 48 and a mixer unit boss 50.

FIG. 8 shows the mixing apparatus 10 fully assembled. The next stage is to introduce the components of bone cement 12 into the mixing cylinder 18. To facilitate this the second drive motor 36 is actuated to rotate the lead screw 34 in a first direction to cause the coupling 32 to translate down the lead screw 34, thereby moving the mixing mechanism 18 away from a mixing cylinder housing inlet 54 which is shown in FIG. 8 covered by a cap 56. The coupling 32 travels along the lead screw 34 until it reaches the lower limit switch 48 which stops further movement of the coupling 32 in that direction. The lower limit switch 48 is positioned to stop the coupling 32 in a position which relates to the mixing mechanism head 26 being at an end of the mixing cylinder 18.

Referring to FIG. 9, the cap 56 is removed and the components of bone cement 12 are introduced into the mixing cylinder 18.

Referring to FIGS. 10, 11 and 12 the motors 30, 36 drive the mixing mechanism 22 backwards and forwards through the components of bone cement 12 mixing it through rotation and axial translation of the mixing mechanism head 26. The mixing head 26 is designed to maximise the mixing effect. The head 26 which is shown in FIG. 3, is essentially a disc 82 defining apertures 84 and having a disc edge 86 defining recesses or openings 88. Rotation and axial movement of the mixing mechanism 22 through the components of bone cement 12 forces the components 12 through the disc apertures 84 and recesses 88, mixing the components 12 into a homogeneous cement of low porosity.

Referring briefly to FIG. 10, the upper extent of travel of the mixing mechanism head 26 is determined by the upper limit switch 46, the upper and lower limit switches 46, 48 determining the sweep of the mixing mechanism 22 in the mixing cylinder 18.

Once the components of bone cement 12 are thoroughly mixed, the mixing mechanism 22 is moved to the position of the lower limit switch 46 and the mixing cylinder 18 is removed from the mixer unit 14.

The cement filled mixer cylinder 18 can then be fitted with a nozzle 90, the nozzle 90 replacing the cap 56, to allow cement to be dispensed through the mixing cylinder inlet 54. In this embodiment the shaft 24 is broken away from the head 26 to allow the mixing cylinder 18 to be attached by the mixing cylinder boss 48 to a dispensing gun (not shown).

Various modifications and improvements may be made to the above-described embodiment without departing from the scope of the invention.

For example, each motor could be provided with an individual battery.

1. A bone cement mixing apparatus for mixing components of bone cement, the apparatus comprising:
a mixer unit, the mixer unit having a drive mechanism; and
a mixing cylinder comprising a housing and a mixing mechanism, the mixing mechanism adapted, in use, to mix components of bone cement contained within the housing;
wherein the drive mechanism is adapted to create rotational movement of the mixing mechanism with respect to the mixing cylinder housing and to create axial movement of the mixing mechanism with respect to the mixing cylinder housing.

2. The mixing apparatus of claim 1, wherein the drive mechanism comprises a first drive motor and a second drive motor, the first drive motor being configured to create rotational movement of the mixing mechanism with respect to the mixing cylinder housing and the second drive motor being adapted to create axial movement of the mixing mechanism with respect to the mixing cylinder housing.

3. The mixing apparatus of claim 1, wherein the mixing mechanism comprises a shaft and a head.

4. The mixing apparatus of claim 3, wherein the head comprises a disc, the disc being mounted at right angles to a first end of the shaft.

5. The mixing apparatus of claim 3, wherein the head and the shaft are rotationally fixed.

6. The mixing apparatus of claim 3, wherein the mixing mechanism is adapted to rotate about a shaft longitudinal axis.

7. The mixing apparatus of claim 6, wherein the mixing mechanism is adapted to move axially in a direction parallel to the shaft longitudinal axis.

8. The mixing apparatus of claim 4, wherein the head defines a plurality of apertures or openings and wherein at least some of the openings are defined by an edge of the disc.

9. (canceled)

10. The mixing apparatus of claim 3, wherein a mixing mechanism shaft is attached at a second end to the drive mechanism.

11. The mixing apparatus of claim 10, wherein the drive mechanism is adapted to rotate the mixing mechanism shaft.

12. The mixing apparatus of claim 11, wherein where the drive mechanism comprises a first drive motor and a second drive motor, the first drive motor being configured to create rotational movement of the mixing mechanism with respect to the mixing cylinder housing and the second drive motor being adapted to create axial movement of the mixing mechanism with respect to the mixing cylinder housing, and the first drive motor rotates the mixing mechanism shaft; and
wherein the second drive motor is adapted to rotate a threaded bar, the threaded bar defining a threaded profile.

13. (canceled)

14. The mixing apparatus of claim 12, wherein the threaded bar cooperatively attaches to a coupling defining a complimentary threaded profile to the threaded bar, and wherein the threaded bar is rotationally fixed with respect to the coupling and adapted to cooperate with coupling such that rotational movement of the threaded bar creates linear movement of the coupling along the threaded bar.

15. (canceled)

16. The mixing apparatus of claim 2, wherein the mixing apparatus comprises limiting means adapted to limit the axial movement of the mixing mechanism within the mixing cylinder.

17. The mixing apparatus of claim 16, wherein the mixing mechanism is adapted to actually translate between two predetermined locations in the mixing cylinder.

18. The mixing apparatus of claim 17, wherein a first predetermined location is proximal the mixer unit and a second predetermined location is distal the mixer unit, and wherein in travelling between the first predetermined location on the second predetermined location, mixing mechanism is adapted to rotate multiple times.

19. (canceled)

20. (canceled)

21. The mixing apparatus of claim 2:
wherein the mixer unit is electrically powered by a battery,
wherein the drive mechanism comprises a first drive motor and a second drive motor, the first drive motor being configured to create rotational movement of the mixing mechanism with respect to the mixing cylinder housing and the second drive motor being adapted to create axial movement of the mixing mechanism with respect to the mixing cylinder housing; and
wherein where the drive mechanism comprises a first and second drive motor, wherein at least one of the drive motors is battery powered.

22-29. (canceled)

30. A mixing apparatus for mixing components of bone cement, the apparatus comprising:
a mixer unit, the mixer unit having at least one motor; and
a mixing cylinder comprising a housing and a mixing mechanism, the mixing mechanism adapted to mix components of bone cement contained within the housing, the at least one motor being adapted to create relative movement between the housing and the mixing mechanism;
wherein the at least one motor is battery-powered.

31. A method of mixing components of bone cement, the method comprising the steps of:
attaching a mixing cylinder to a mixer unit;
introducing components of bone cement to the mixing cylinder;
actuating the mixer unit to mix the components of bone cement by creating relative movement in rotational and axial direction of a mixing cylinder mixing mechanism with respect to a mixing cylinder housing; and
removing the mixing cylinder from the mixer unit.

32. The method of claim 31, wherein the step of actuating the mixer unit to mix the components of bone cement by creating relative movement in rotational and axial direction of a mixing cylinder mixing mechanism with respect to a mixing cylinder housing further comprises the steps of:
actuating a first mixer unit drive motor to create rotational movement of the mixing cylinder mixing mechanism with respect to the mixing cylinder housing; and
actuating a second mixer unit drive motor to create axial movement of the mixing cylinder mixing mechanism with respect to the mixing cylinder housing.

33. The method of claim 31, wherein the step of attaching the mixing cylinder to the mixer unit may further comprise the steps of:
attaching a mixing cylinder mixing mechanism shaft to a mixer unit drive mechanism; and
attaching a mixing cylinder housing to a mixer unit body.

34. (canceled)

35. (canceled)
